(19) United States
(12) Patent Application Publication
(10) Pub. No.: US 2026/0141485 A1
(43) Pub. Date: May 21, 2026

(54) METHOD AND DEVICE FOR ENHANCING X-RAY CAPTURES

(71) Applicant: Siemens Healthineers AG, Forchheim (DE)

(72) Inventors: Felix Denzinger, Herzogenaurach (DE); Elisabeth Preuhs, Erlangen (DE); Sebastian Käppler, Nürnberg (DE)

(21) Appl. No.: 19/394,866

(22) Filed: Nov. 19, 2025

(30) Foreign Application Priority Data
Nov. 20, 2024 (DE) 10 2024 211 127.9

(51) Int. Cl. G06T 5/50; A61B 6/00; A61B 6/50; G06T 5/60; G06T 5/70; G06T 7/20; G06T 7/33; G06T 7/73; G06V 10/26

(52) U.S. Cl. CPC G06T 5/50; A61B 6/485; A61B 6/503; A61B 6/5205; A61B 6/5258; A61B 6/54; G06T 5/60; G06T 5/70; G06T 7/20; G06T 7/337; G06T 7/74; G06V 10/26; G06T 2207/10121; G06T 2207/20081; G06T 2207/20092; G06T 2207/20216; G06T 2207/30168; G06V 2201/03

(57) ABSTRACT

A method for enhancing X-ray captures includes providing a series of X-ray captures that are temporally successive captures of a same region of interest. The X-ray captures of the series are segmented by identifying structures in the region of interest and determining a certainty value for identified structures that indicates identification quality. At least some of these structures as reference structures based on their certainty value. The X-ray captures of the series are registered on one another. The reference structures are registered on one another in accordance with a registration function. A result image is created by linking image information in the registered X-ray captures, and the result image is output.

This application claims the benefit of German Patent Application No. DE 10 2024 211 127.9, filed on Nov. 20, 2024, which is hereby incorporated by reference in its entirety.

### BACKGROUND

The present embodiments relate to enhancing X-ray captures.

In X-ray imaging (e.g., fluoroscopic imaging), the depiction of structures such as disease processes, anatomical points of reference, or medical objects that are of relevance from an anatomical or processing standpoint is normally degraded by noise. This is attributable to the “as-low-as-reasonably-achievable” (ALARA) principle because, for the wellbeing of the patient, physicians are keen to keep the radiation dose as low as possible. However, this increases image noise. In addition to spatial noise suppression, time-averaging of the image information is a conventional approach to reducing image noise. However, if the structure of interest moves between the images, the structure becomes somewhat blurred on averaging rather than being accentuated.

One possibility for countering this is to register a number of images before the images are averaged over the time domain. This, however, requires the structures to be visible over a plurality of successive frames. One example of this is an algorithm in which balloon marker pairs are tracked over a plurality of frames and then registered on one another in order to obtain a sharper view of an object such as, for example, a stent. This makes it possible to assess whether the object has been successfully placed. Balloon markers are strongly X-ray-absorbing thickened portions on a guide wire that is pushed through a catheter. Balloon markers are located, for example, where there is a stent balloon.

No method that is applicable to larger or a plurality of structures has previously existed.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method and a device for enhancing X-ray captures, a control facility for a medical technology system, and a medical technology system that overcome the above-described disadvantages are provided.

A method according to the present embodiments serves to enhance X-ray captures. These X-ray captures may be fluoroscopy captures. The method includes the following acts: providing a series of X-ray captures that represent temporally successive captures of the same region of interest; segmenting the X-ray captures of the series by identifying structures in the region of interest and determining a certainty value for identified structures that indicates identification quality; selecting at least some of these structures as reference structures based on their certainty value; registering the X-ray captures of the series on one another, where the reference structures are registered on one another in accordance with a registration function; creating a result image by linking image information in the registered X-ray captures; and outputting the result image.

The method for enhancing X-ray captures may, for example, be used for noise suppression or in general for signal amplification. The sequence of the method for enhancing X-ray captures may be described as follows.

First, a series of temporally successive X-ray captures of the same region of interest (ROI) are created. These may be, for example, a series X-ray images of a moving object, such as a moving organ(e.g., a heart) during an examination. Provision may include capturing the X-ray captures as well as downloading them from a database.

Next, structures within the captures are identified and delimited from other structures (e.g., segmented). This may be carried out manually by an expert or, for example, automatically by dedicated software. For this purpose, the structures may be recognizable, at least for the entity carrying out the method, on the images over a plurality of successive frames. Image segmentation is well known per se in the prior art. The heart, aorta, coronary vessels, and a stent may, for example, be segmented in the X-ray captures. In one embodiment, two or more structures in the X-ray captures are segmented.

The term “segmenting” also includes landmark detection that may subsequently be used together with an orientation calculation for registration. In landmark detection, specific known points of the structures in the X-ray captures are recognized as landmarks, and the structures are segmented in this way by defining prominent points. Landmark detection may well be used for registration without further segmentation (e.g., by placing the landmarks per se on one another). It is, however, also possible to use the landmarks of specific structures or regions in order to segment them still better.

A certainty value for identified structures that indicates identification quality is additionally determined. When segmenting via a trained machine learning-capable system, this may be achieved, for example, by reading out its probability values for the segmented image regions. The certainty value may, however, also be determined subsequently in the course of checking the segmentation. It is merely important that, for each of the identified (e.g., and thus segmented) structures, a value that indicates a measure of identification quality (e.g., or segmentation) and/or indicates a measure of the completeness of the segmented structure is available. For ease of understanding, it may be assumed that this value is between 0 and 1 and its level is a measure of quality.

At least one of these identified structures is selected as a reference in order to register the captures on one another. For ease of understanding, these structures are denoted reference structures but constitute at least some (e.g., all) of the segmented structures. The reference structures may be present in all the X-ray captures since registration is otherwise made more difficult. There is, however, also a solution in the event that reference structures are not visible in all of the X-ray captures. This involves dividing the series of X-ray captures into subseries and is described more precisely below.

The reference structures are selected based on their certainty values. It is possible to predefine how precisely this is to take place. The structures selected may be those having certainty value that indicates the highest quality (e.g., those structures with the highest certainty value). A range of values may well be predetermined, and all the structures lying within this range of values may be selected as reference structures. It is, however, also possible to predetermine a number N of structures and to select the N structures having certainty values that indicate the highest quality. It is also possible for those structures having certainty value that indicates the highest quality to be proposed to a user who then selects the reference structures.

The individual X-ray captures are then registered on one another using the selected reference structures and an appropriate mathematical function (e.g., registration function) in order to make the X-ray captures optimally comparable with one another at least in the region of the reference structures. At least the region of the reference structures is registered here. The region of the X-ray captures extending therebeyond may also be registered (e.g., according to the same registration function) or may remain unregistered.

Not all the regions of an X-ray capture are always of medical interest. It may well be that just one organ or medical object is to be observed. This organ or object may then be part of the reference structures, and it is not necessary to register, for example, the edges of the X-ray captures on one another. For example, the positions of the heart, the surrounding vessels, and an artificial heart valve are brought into line with one another with the assistance of the registration function. The principle of image registration per se and the creation of a registration function are known in the prior art.

The remaining regions of the X-ray captures (e.g., outside the reference structures) may thus also be registered on one another according to this registration function. This is, however, not absolutely necessary. The method may be configured in this respect such that this possibility may be selected or deselected via a user interface. It may even be that the regions outside the segmented structures are to be left unchanged and registration is deliberately to be carried out only in an overlay region (e.g., in an ellipse). It may be preferred for a structure under examination to be averaged over the time dimension with a certain surrounding “halo.” In this case too, registration is to be performed only in this region.

The registration function may be the same across the reference structures or alternatively be different in different regions (e.g., for different reference structures).

A result image is then created based on the registered X-ray captures. A plurality of result images may be created, but at least one result image is to be created. This result image is to be based on a plurality of registered X-ray captures. This provides that the result image has been created using the image information from a plurality of registered X-ray captures. In one embodiment, each pixel of the result image may be based on the corresponding pixels of the respective registered X-ray captures (e.g., in each case at the same image coordinates). This may take place, for example, by the image values in question of the registered X-ray captures being summed, subtracted from one another, multiplied by one another, or divided by one another. Basically, all modern image processing possibilities are available.

The one or more created result images are finally output. Thanks to the use of a plurality of captures registered on one another for one result image, this result image has an enhanced quality and accuracy in comparison with the original X-ray captures if the method is correctly applied. This output of the result images may be used for assessing the number of result images on a display, or the output of the result images may simply be stored.

In practice, the following procedure may be provided: First, the frames to be registered (e.g., the X-ray captures) may be segmented, and then, the structures (e.g., reference structures) to be used for registration are selected. If the selected structures are no longer usable, the selection of reference structures is modified. Registration then takes place, followed by creation of the result image.

In order to make use of as many structures that are depicted in the frames as possible, a general segmentation method is used that segments a large quantity of structures in each frame according to known automated methods. Various methods may be used. For example, segmentation algorithms that differentiate between foreground and background in order to select only important structures (e.g., from the foreground) may be used. Further, various features of the (e.g., segmented) structures may be segmented or recognized in order to assist with the subsequent registration. Specific points of reference, contrast features, metrics, or edge information may be recognized for this purpose and may then (e.g., optionally additionally) be used for registration. Segmentation quality need not be perfect (e.g., if the center point of the structure of interest and its orientation are of most significance for the subsequent steps).

Only the well segmented structures may be selected for registration. In one embodiment, for this purpose, the segmentation method may output a value (e.g., “certainty value”) for each of the segmented structures that reflects the level of certainty about how correctly the structure is segmented and recognized. For example, the certainty value indicates how certain it is that a structure recognized as a “heart” also actually does depict the heart. Determining such a value is prior art and is not explained further here. This value may, for example, be determined together with the segmentation or in the course of subsequent recognition. Segments that are to be examined in greater detail in the course of an examination may now be selected, but it is also possible to select the N (e.g., integer>0) structures with the highest certainty values. However, a (e.g., single) structure to be examined may also be selected and additionally N further structures. This may be achieved, for example, by using Monte Carlo dropout with deep learning-based methods.

Registration may be carried out using various (e.g., deep learning-based) methods in order to provide a real-time runtime (see, e.g., Fu, Y. et al. “Deep learning in medical image registration: a review. Physics in Medicine & Biology,” 65(20), 20TR 01;2020). A further method may involve calculating the centroid with a clear main axis on each frame and making a comparison between the frames, and establishing a resultant relation between the frames in order to obtain the relationship between them. A way of enhancing robustness of the present embodiments is to focus solely on the position and orientation of the structures of interest. Alternatively, template matching may be used to carry out robust registration.

If a selected structure may no longer effectively be segmented in the frames or disappears from the image region (or “field of view”) (e.g., due to an anatomical movement or C-arm movement), it is possible to segment new structures for registration. One possibility would be to replace the old structure with the next most suitable, or to select a new reference structure. The registration itself is retained by the other structures, which were used previously, as well as those that are no longer used. If this is not possible, the user may be notified that the reference structures between two frames are no longer present, and manual registration by the user is to be provided. Once this has been handled, a search for new structures for registration may be performed.

In one embodiment, in clinic practice, a user may additionally be able to determine how many structures are to be used for registration between two frames (e.g., between 1 and 10). It is further provided for the user to be able to determine the anatomical region from which the structures for registration are to be used (e.g., only from a specific vessel or vessel region). It is advantageous for this purpose for the current segmentation of the structures together with the certainty value to be displayed. The user may then determine which structures are to be used (e.g., by clicking thereon or drawing an outline). In one embodiment, the user may be able to determine that only structures with a specific certainty value (e.g., >95%) are to be used for registration.

Speech control may be used for user inputs.

One advantage of the method is that the method offers a flexible, complete solution in which various structures of interest may be enhanced depending on the image and input from a physician. This enables flexible use in a number of different scenarios, where only one single component within a product is to be validated or borne in mind. Further, simultaneous use may be made of a plurality of structures that may boost performance. Further, there is no limitation to individual structures, as they can instead be dynamically adapted.

A device according to the present embodiments serves to enhance X-ray captures. The device includes the following components: a data interface configured to receive or retrieve a series of X-ray captures that represent temporally successive captures of the same region of interest; a segmentation unit configured to segment the X-ray captures of the series by identifying structures in the region of interest and to determine a certainty value that indicates identification quality; a selection unit configured to select at least some of these structures as reference structures based on their certainty value; a registration unit configured to register the X-ray captures of the series on one another, where the reference structures are registered on one another in accordance with a registration function; a result unit configured to create a result image by linking image information in the registered X-ray captures; and a data interface configured to output result images.

The device may be configured to carry out a method according to the present embodiments and, for example, permits enhancement of X-ray captures. The device includes a plurality of interconnected components that, in interplay, enable high quality imaging.

The first component is the data interface that is configured to receive or retrieve a series of temporally successive X-ray captures of the same region of interest. One example of this would be importing a sequence of X-ray images of a moving object.

These images are then forwarded to the segmentation unit that identifies and segments the structures within the X-ray captures. A certainty value for identified structures that indicates identification quality is additionally determined.

The selection unit then selects at least some of these identified structures as reference structures based on their certainty value (e.g., see above description relating to the method). These reference structures are decisive for the subsequent registration of the captures on one another.

The registration unit then registers the selection of X-ray captures on one another by registering the reference structures on one another in accordance with a registration function and may also correspondingly adapt at least some of the other regions of the captures. One example of this would be aligning X-ray images of a moving heart by using the structure of the pericardium and the surrounding vessels as reference structures.

The result unit serves to create a result image by linking image information in the registered X-ray captures. This has already been described in the context of the method. A denoising unit may optionally be used as the result unit in order to create a noise-suppressed or signal-optimized image. This may be achieved by averaging (e.g., time-averaging) the registered X-ray captures. For example, the denoising unit may reduce the noise in a sequence of X-ray images by calculating the average intensity of each pixel over all the captures.

Finally, a further data interface outputs the registered X-ray captures or, if available, the noise-suppressed image. This makes it possible to display the enhanced images on a monitor or to store the enhanced images for further analysis.

A control facility according to the present embodiments for a medical technology system (e.g., a diagnostic system or an X-ray system, such as a fluoroscopy system) includes a device according to the present embodiments and/or is configured to carry out a method according to the present embodiments.

A medical technology system according to the present embodiments may be a diagnostic system or an X-ray system (e.g., a fluoroscopy system) and includes a control facility according to the present embodiments.

The present embodiments may, for example, be implemented in the form of a computer unit with suitable software. The computer unit may have, for example, one or more cooperating microprocessors or the like. For example, the computer unit may be implemented in the computer unit in the form of suitable software program parts. A largely software-based implementation has the advantage that computer units that are already in use may also straightforwardly be upgraded to operate in the manner according to the present embodiments using a software or firmware update. In this respect, the object is also achieved by a corresponding computer program product with a computer program that is directly loadable into a storage device of a computer unit, with program parts for carrying out all the acts of the method according to the present embodiments when the program is executed in the computer unit. In addition to the computer program, such a computer program product may optionally include additional elements such as, for example, documentation and/or additional components including hardware components, such as, for example, hardware keys (e.g., dongles, etc.) for using the software.

A computer-readable medium (e.g., a non-transitory computer-readable storage medium, such as a memory stick, hard disk, or other transportable or permanently installed data storage medium), on which the program parts of the computer program that may be read in and executed by a computer unit are stored, may be used for transport to the computer unit and/or for storage on or in the computer unit (e.g., a computer).

Further, refinements and further developments of the present embodiments are revealed below, where one category may also be further developed in a manner similar to the passages of the description relating to another category. For example, individual features of different example embodiments or variants may also be combined to form new example embodiments or variants.

The result image may be a noise-suppressed or signal-amplified image that was created based on a plurality of X-ray captures by averaging registered X-ray captures. It is not necessary to use all the registered X-ray captures for this purpose, but it may well be advantageous. However, in the event that a plurality of result images are to be produced in the form of an image series, only some of the registered X-ray captures may be used for one result image. For example, in the case of N registered X-ray captures, in each case M<N successive registered X-ray captures may be used for one result image, where captures 1 to M are used for the first result image and captures 2 to (M+1) for the second, and so on.

Result images may be created by averaging the registered X-ray captures. This provides that the image values of corresponding image coordinates are averaged. Since the X-ray images are temporally successive, time-averaging may be provided. However, this basically provides that corresponding image values of temporally successive captures are averaged. Averaging may, for example, provide that N image values of N registered X-ray captures are summed and normalized (e.g., by dividing by N).

The method may include the acts of: creating a noise-suppressed image as the result image based on the registered X-ray captures, such as by time-averaging the registered X-ray captures; and outputting the noise-suppressed image as the result image.

The method may include the acts of: creating a signal-optimized image as the result image based on the registered X-ray captures (e.g., by time-averaging the registered X-ray captures); and outputting the signal-optimized image as the result image.

Image denoising is basically known per se. The peculiarity in this case, however, is that registration of the X-ray captures by the method permits particularly good image denoising in the region of the reference structures.

Time-averaging may, for example, simply be achieved by summing the image values of the same image coordinates of the X-ray captures and normalizing them all with the same value (e.g., the number of images).

At least two structures may be identified and defined as reference structures. In order to make use of as many structures that are depicted in the frames (e.g., X-ray captures) as possible, a general segmentation method that segments a large quantity of structures in each frame is used. In one embodiment, segmentation algorithms may be used that differentiate between foreground and background in order to select only important structures (e.g., from the foreground). A user may determine how many and/or which structures are to be used for image registration (e.g., 1 to 10). This information may also take the form of defaults. Alternatively or additionally, a user may determine the anatomical region from which structures are to be used (e.g., only from a specific vessel or from a specific vessel region). This information may also take the form of defaults.

According to an embodiment of the method, segmentation is carried out such that various features of the segmented structures are identified. “Features” are taken to be derived properties of the segmented regions (e.g., prominent points of an organ or its size). Registration may then be carried out based on these features. In one embodiment, the features may include one or more predetermined components of the group of points of reference, contrast features, metrics, average values, and edge information.

Segmentation quality need not be perfect since it is the center point of the structure of interest and its orientation that are of most significance for the subsequent acts. Segmentation may therefore merely involve prominent landmarks of an organ and its orientation in space. During registration, the landmarks may then be registered on one another, and all the interposed points in the X-ray captures are registered based on the registration of the bounding landmarks.

In one embodiment of the method, the selection of reference structures may be based on a certainty value, and a number of those segmented structures with the greatest certainty may be selected as reference structures. The number is predetermined, or those structures with a certainty value above a specified limit value are selected as reference structures. The term “certainty value” has already been explained above. The determination of this value in the context of image segmentation or subsequent image recognition is basically known. In the simplest case, the certainty value indicates the probability for each pixel in a structure that a pixel is part of this structure. The average of all the pixel probabilities of a region may, for example, then be calculated and used as the certainty value for the region (e.g., how certain the network is that a pixel is also part of the structure thereof).

In one embodiment, the certainty value may be determined via Monte Carlo dropout with deep learning-based methods.

The segmented ranges may be displayed to a user together with the certainty value, and the user may then select segments (e.g., by clicking with a mouse on the structure). It may be defined that only structures with a certainty value above a specified limit value are to be used. While structures having those certainty values that indicate the highest quality may be selected, a user may prefer other structures.

In one embodiment, a deep learning-based method may be used for registration in order to provide a real-time runtime. Alternatively or additionally, a calculation of the centroid with a main axis on each X-ray capture may be provided. Alternatively or additionally, a comparison of the results between the X-ray captures may be provided in order to obtain the relationship between them.

In one embodiment, the position and orientation of the reference structures may be taken into account (e.g., exclusively) for registration.

In one embodiment, template matching may be used for registration. This is a method in image processing and pattern recognition, in which a given image (e.g., the template) is compared with part of a larger image in order to identify similarities or matches. The aim of template matching is to identify the positions in the image where the template fits best. The “template matching” principle is known in the prior art and may enhance robustness.

According to an embodiment of the method, a quality metric that indicates a measure of the quality of registration of the reference structures on one another or indicates a measure of a difference between reference structures of the same kind is formed. Determination of the quality metric is known in the prior art in the context of determining image registration quality. If the quality metric is outside a specified quality range, the series of X-ray captures is divided into a plurality of subseries of X-ray captures, where individual reference structures are selected for each subseries, and the method is continued for each subseries as a self-contained series.

If the selected structures are no longer usable, the selection of reference structures may be modified (e.g., by replacing an existing reference structure with another structure of a segmented X-ray capture as a new reference structure).

If automatic segmentation or registration is not possible, manual segmentation or manual registration may be carried out by a human for one subseries.

According to an embodiment of the method, a quality metric is derived from how well a selected structure may be segmented or whether a structure disappears from the image region (e.g., due to an anatomical movement or C-arm movement).

In one embodiment, a change vector may be derived from a change in (e.g., a movement of) a structure in the series of X-ray captures, and this change vector may be used for registration. In this respect, a structure may well move on a clearly determinable trajectory. This trajectory may be determined by external parameters (e.g., on movement of a C-arm or by parameters that may be derived from the individual X-ray captures, such as on movement of an object through the bloodstream). If a trajectory of an object, or even the trajectories of a plurality of landmarks of an object, is/are known, it is then possible to extrapolate a change in the object from the trajectories even if the object is not visible. This extrapolated change may also be used for image registration.

According to an embodiment of the method, subseries of X-ray captures are registered on one another based on the same reference structures in both subseries. Should this not be possible, a user may be notified that the registration structures between two frames are no longer present, and manual registration by the user is to be provided.

One embodiment of a device includes at least one machine learning-capable model that has been appropriately trained for its task. This is, for example: a machine learning-capable model that has been trained to segment X-ray captures; and/or a machine learning-capable model that has been trained to register X-ray captures; and/or a machine learning-capable model that has been trained to select reference structures. This may well be configured for image recognition or to determine suitable organs from an examination specification.

Components of the present embodiments may take the form of a “cloud service.” Such a cloud service serves to process data (e.g., using artificial intelligence), but may also be a service based on conventional algorithms or a service in which interpretation is provided in the background by humans. In general, a cloud service (hereinafter also denoted “cloud” for short) is IT infrastructure in which, for example, storage space or computing power and/or application software is provided across a network. Communication between the user and the cloud, for example, proceeds via data interfaces and/or data transfer protocols. In the present case, the cloud service may provide both computing power and application software.

In the course of a method of the present embodiments, data that is obtained in the course of the present embodiments is provided via the network to the cloud service. The cloud service includes a computer system that may not include the local computer of the user. In one embodiment, the method may be implemented using a command constellation in a network. The data calculated in the cloud is subsequently sent back via the network to the local computer of the user.

### BRIEF DESCRIPTION OF THE DRAWINGS

The invention is explained again in greater detail below based on example embodiments with reference to the appended figures. In the various figures, same components are provided with same reference signs. Generally, the figures are not to scale. In the figures:

FIG. 1 shows an example of an X-ray system with a device or control facility according to the present embodiments;

FIG. 2 shows a sequence of an example embodiment of a method according to the present embodiments;

FIG. 3 shows one possibility for forming a subseries of X-ray captures;

FIG. 4 shows an alternative possibility for forming a subseries of X-ray captures and registering the subseries of X-ray captures.

### DETAILED DESCRIPTION

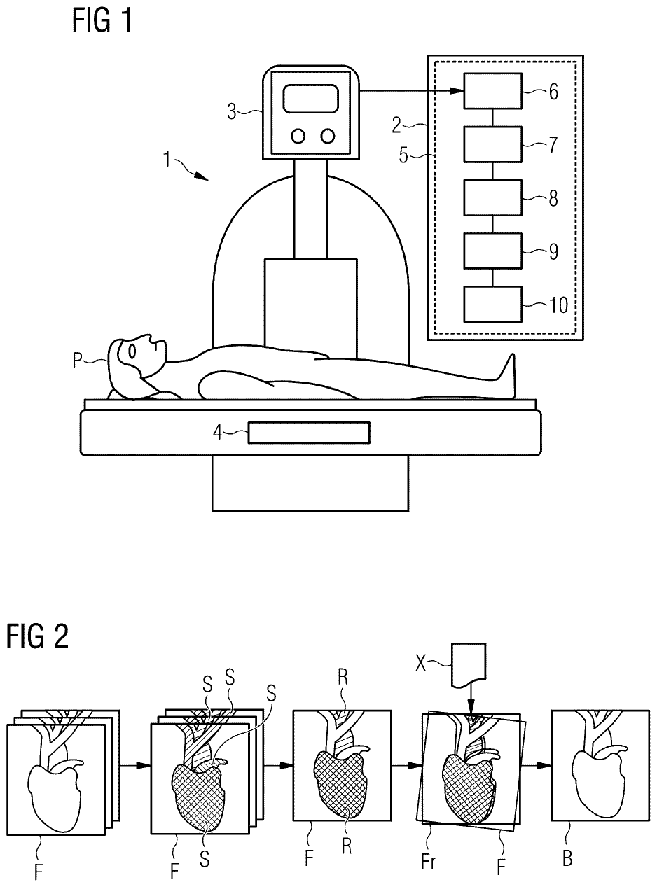

FIG. 1 shows a rough schematic of an X-ray system 1 as an example of a system for taking X-ray captures F (see also FIG. 2) with a control facility 2. The control facility 2 (e.g., including one or more processors) is equipped with a device 5 configured to carry out the method according to the present embodiments.

The X-ray system 1 has, as is conventional, a radiation source 3 that, in the present case, represents an X-ray source, and during an X-ray capture F, irradiates a body region of a patient P, such that the radiation impinges on a detector 4 that is in each case located opposite the radiation source 3.

Only those components that are essential for explaining the present embodiments are depicted in the control facility 2. X-ray systems 1 do not, in principle, need to be explained in detail.

The control facility 2 includes a device 5 for enhancing X-ray captures F via a corresponding method, as is, for example, depicted in FIG. 2, and includes a data interface 6, a segmentation unit 7, a selection unit 8, a registration unit 9, and a denoising unit 10 as the result unit 10.

The data interface 6 serves to receive or retrieve a series of X-ray captures F that represent temporally successive captures of the same region of interest. The data interface 6 additionally serves to output the registered X-ray captures F or, for example, if available, the noise-suppressed image B.

The segmentation unit 7 serves to segment the X-ray captures F by identifying structures S in the region of interest.

The selection unit 8 serves to select at least some of these structures S as reference structures R based on their certainty value.

The registration unit 9 serves to register the X-ray captures F on one another, where the reference structures R are registered on one another in accordance with a registration function X.

The denoising unit 10 (e.g., result unit 10) serves to create a noise-suppressed image B (e.g., by time-averaging the registered X-ray captures F, Fr).

FIG. 2 shows from left to right a sequence of an example embodiment of a method for enhancing X-ray captures F.

First (on the left), a series of X-ray captures F is provided (e.g., after capture by the X-ray system according to FIG. 1). These X-ray captures F are a series of temporally successive captures of the same region of interest.

The X-ray captures F of the series are then segmented by identifying structures S in the region of interest (e.g., second image from the left).

After segmentation, some of these structures S are selected as reference structures R (e.g., third image from the left). In the example shown, the heart and aorta have been selected as reference structures R.

The X-ray captures F of the series are then registered on one another, where the reference structures R are registered on one another in accordance with a registration function X. One of the X-ray captures F may remain unchanged, and the other X-ray captures Fr are registered on the one X-ray capture. The “registered X-ray captures” F, Fr are then the one unchanged X-ray capture F and the other registered X-ray captures Fr.

These registered X-ray captures F, Fr may then be output. In the case depicted in Figure, a denoised image B produced from the registered X-ray captures F, Fr, and the image B is then output (e.g., on the right).

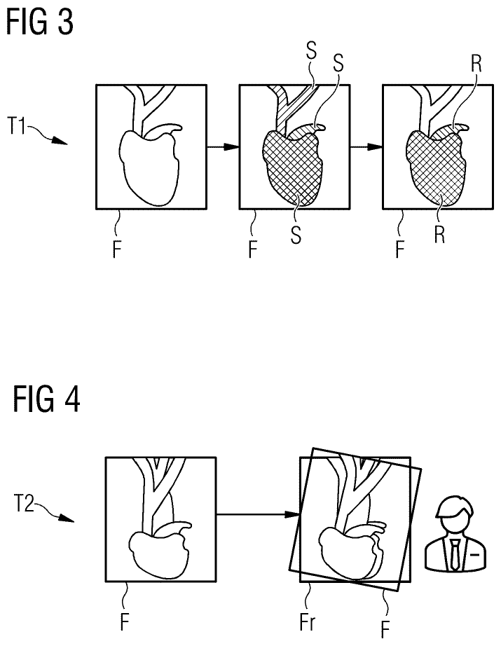

FIG. 3 shows one possibility for forming a subseries T1 of X-ray captures F. It was the case, for example, that the aorta was no longer recognizable in some of the X-ray captures F (cf., FIG. 2). Other structures S were thus selected as reference structures R for a subseries T1. Registration, which corresponds to what is shown in FIG. 2, may now be carried out using these reference structures.

FIG. 4 shows an alternative possibility for forming a subseries T2 of X-ray captures and the registration thereof. In this case, the structures S are deformed to such an extent that an automated procedure does not produce satisfactory results. Image registration is thus performed by a human.

It may, for example, be checked whether registration on one another is still possible at the (e.g., temporal) edges of subseries T1, T2. In this case, subseries T1, T2 may then be registered on one another.

The present embodiments described in detail above merely involve example embodiments that may be modified in the most varied manner by a person skilled in the art without departing from the scope of the invention. Further, use of the indefinite article “a” does not rule out the possibility of a plurality of the features in question also being present. Likewise, terms such as “unit” do not rule out the possibility of the components in question consisting of a plurality of interacting subcomponents that may optionally also be spatially distributed. The term “a number” should be taken to be “at least one.” Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for enhancing X-ray captures, the method comprising:
providing a series of X-ray captures that represent temporally successive captures of a same region of interest;
segmenting the X-ray captures of the series, the segmenting comprising identifying structures in the region of interest and determining a certainty value for identified structures that indicates identification quality;
selecting at least some of the structures as reference structures based on the certainty values, respectively;
registering the X-ray captures of the series on one another, wherein the reference structures are registered on one another in accordance with a registration function;
creating a result image, the creating of the result image comprising linking image information in the registered X-ray captures; and
outputting the result image.

2. The method of claim 1, wherein the result image is a noise-suppressed or signal-amplified image, and
wherein creating the result image comprises averaging the registered X-ray captures.

3. The method of claim 1, wherein the segmenting is carried out such that various features of the segmented structures are identified, and the registering is carried out based on the various features, and
wherein the various features comprise one or more predetermined components, the one or more predetermined components comprising points of reference, contrast features, metrics, average values, edge information, or any combination thereof.

4. The method of claim 1, wherein the selecting of the reference structures comprises selecting a number of the segmented structures having certainty values that indicate a highest quality, and
wherein the number is predetermined or the structures with the certainty value above a specified limit value are selected as the reference structures.

5. The method of claim 4, wherein the certainty value is determined via Monte Carlo dropout with deep learning-based methods.

6. The method of claim 1, wherein the registering comprises registering the X-ray captures of the series on one another using a deep learning-based method, calculation of a centroid with a main axis on each of the X-ray captures, a comparison and resultant relation between the X-ray captures, or any combination thereof.

7. The method of claim 1, wherein:
a position and an orientation of the reference structures are taken into account for the registering;
template matching is used; or
a combination thereof.

8. The method of claim 7, wherein the position and the orientation of the reference structures are taken into account exclusively for the registering.

9. The method of claim 1, further comprising:
forming a quality metric that indicates a measure of quality of the registration of the reference structures on one another or indicates a measure of a difference between reference structures of the same kind; and
when the quality metric is outside a specified quality range:
dividing the series of X-ray captures into a plurality of subseries of X-ray captures, wherein individual reference structures are selected for each subseries of the plurality of subseries; and
the method is continued for each subseries of X-ray captures of the plurality of subseries of X-ray captures as a self-contained series.

10. The method of claim 9, wherein manual segmentation, manual registration, or manual segmentation and manual registration are carried out by a human for one subseries of X-ray captures of the plurality of subseries of X-ray captures.

11. The method of claim 9, wherein the quality metric is derived from:
how well a selected structure is segmentable; or
whether a structure disappears from an image region.

12. The method of claim 11, further comprising deriving a change vector from a change in a structure in the series of X-ray captures,
wherein the change vector is used for the registering.

13. The method of claim 12, wherein the change in the structure is a movement of the structure.

14. The method of claim 9, wherein subseries of the plurality of subseries of X-ray captures are registered to one another based on same reference structures in both of the subseries.

15. A device for enhancing X-ray captures, the device comprising:
a data interface configured to receive or retrieve a series of X-ray captures that represent temporally successive captures of a same region of interest;
a segmentation unit configured to:
segment the X-ray captures of the series, the segmentation comprising identification of structures in the region of interest; and
determine a certainty value that indicates identification quality;
a selection unit configured to select at least some of the structures as reference structures based on the certainty value, respectively;
a registration unit configured to register the X-ray captures of the series on one another, wherein the reference structures are registered on one another in accordance with a registration function;
a result unit configured to create a result image, the creation of the result image comprising linking image information in the registered X-ray captures; and
a data interface configured to output result images.

16. The device of claim 15, wherein the result unit is a denoising unit configured to create a noise-suppressed image.

17. The device of claim 16, wherein the denoising unit is configured to time-average the registered X-ray captures, such that the noise-suppressed image is created.

18. A medical technology system comprising:
a control facility for a medical technology system, the control facility comprising:
a device for enhancing X-ray captures, the device comprising:
a data interface configured to receive or retrieve a series of X-ray captures that represent temporally successive captures of a same region of interest;
a segmentation unit configured to:
segment the X-ray captures of the series, the segmentation comprising identification of structures in the region of interest; and
determine a certainty value that indicates identification quality;
a selection unit configured to select at least some of the structures as reference structures based on the certainty value, respectively;
a registration unit configured to register the X-ray captures of the series on one another, wherein the reference structures are registered on one another in accordance with a registration function;
a result unit configured to create a result image, the creation of the result image comprising linking image information in the registered X-ray captures; and
a data interface configured to output result images.

19. The medical technology system of claim 18, wherein the medical technology system is a diagnostic system or an X-ray system, the X-ray system being a fluoroscopy system.

20. A non-transitory computer-readable storage medium that stores instructions executable by a computer to enhance X-ray captures, the instructions comprising:
providing a series of X-ray captures that represent temporally successive captures of a same region of interest;
segmenting the X-ray captures of the series, the segmenting comprising identifying structures in the region of interest and determining a certainty value for identified structures that indicates identification quality;
selecting at least some of the structures as reference structures based on the certainty values, respectively;
registering the X-ray captures of the series on one another, wherein the reference structures are registered on one another in accordance with a registration function;
creating a result image, the creating of the result image comprising linking image information in the registered X-ray captures; and
outputting the result image.
